(19) United States
(12) Patent Application Publication
(10) Pub. No.: US 2026/0209707 A1
(43) Pub. Date: Jul. 23, 2026

(54) ARTIFICIAL TISSUE TUBE AND METHOD FOR PRODUCING SAME

(71) Applicant: KEIO UNIVERSITY, Tokyo (JP)

(72) Inventors: Hiroaki ONOE, Yokohama-shi, Kanagawa (JP); Shun ITAI, Yokohama-shi, Kanagawa (JP)

(73) Assignee: KEIO UNIVERSITY, Tokyo (JP)

(21) Appl. No.: 19/141,041

(22) PCT Filed: Dec. 19, 2023
(86) PCT No.: PCT/JP2023/045514

§ 371 Date: Jun. 18, 2025

(30) Foreign Application Priority Data
Dec. 21, 2022 (JP) 2022-204999

(51) Int. Cl. C12N 5/077; C12N 5/00; C12N 5/071

(52) U.S. Cl. CPC C12N 5/0661; C12N 5/0062; C12N 5/069; C12N 2513/00

(57) ABSTRACT

[Problem] To provide an artificial tissue tube that can deform more flexibly. [Solution] A smooth muscle layer 11 including smooth muscle coiled in a spiral shape or an annular shape is built in an artificial tissue tube 10. The artificial tissue tube can be produced by a method for producing an artificial tissue tube including a smooth muscle layer creation step in which fibrous smooth muscle tissue is wound around a core rod to produce the smooth muscle layer. The artificial tissue tube has a smooth muscle layer including smooth muscle coiled in a spiral shape or an annular shape.

### TECHNICAL FIELD

The present invention relates to an artificial tissue tube used for applications such as artificial blood vessels and a method for producing the same.

### BACKGROUND ART

Biochemical reactions in blood vessels play a crucial role in immune responses and various diseases. Accordingly, the construction of in vitro vascular models that mimic biological tissues is in high demand in biomedical research and pharmacokinetic testing.

In this context, the present inventors propose the generation of a tubular body that reproduces a vascular structure using an in vitro culture device comprising a multilayered scaffold material (Patent Document 1). The multilayer structure disclosed in Patent Document 1 consists of multiple stacked tubular layers made of extracellular matrix. For example, in the case of reproducing a blood vessel, the tubular body is formed with a two-layer structure using an extracellular matrix such as collagen. Outer membrane cells are seeded on the inside of the first tubular layer, smooth muscle cells are seeded on the inside of the second tubular layer, and endothelial cells are seeded on the inner circumferential surface of the tubular body. This is said to enable the low-cost and simple fabrication of an in vitro culture device with a multilayered scaffold material.

### CITATION LIST

### Patent Literature

Patent Document 1: International Publication Pamphlet No. WO2018/207783

### SUMMARY OF THE INVENTION

### Technical Problem

However, the conventional multilayer structure proposed in Patent Document 1 presents a remaining issue in that, as described above, cellular tissues such as outer membrane cells and smooth muscle cells are seeded onto a rigid scaffold made of collagen or the like and fixed within it, making it difficult to accurately mimic in vivo deformation responses of biological structures such as blood vessels. Specifically, because the scaffold in the conventional multilayer structure is rigid and lacks sufficient flexibility, the contraction of the cellular tissues may be hindered, potentially resulting in behavior that deviates from that of other biological tissues.

Accordingly, a primary object of the present invention is to provide an artificial tissue tube capable of more flexible deformation in order to reproduce the physiological deformation responses of biological structures such as blood vessels.

### Solution to Problem

A first aspect of the present invention relates to an artificial tissue tube. The artificial tissue tube according to the present invention is an artificially created tubular structure containing cellular tissue and can reproduce the contractile and relaxational movements of tubular biological structures such as blood vessels, bronchi, and intestines. The artificial tissue tube of the invention has a smooth muscle layer comprising smooth muscle arranged in a helical or annular pattern. Smooth muscle refers to muscle without sarcomeres (muscle segments) and adjusts the inner diameter of the tubular structure by repeated contraction and relaxation. In the invention, for example, fibrous smooth muscle tissue may be helically wound, and the hollow portion formed by the helical structure may serve as a lumen for blood or the like. Alternatively, multiple rings of fibrous smooth muscle tissue having the same diameter may be prepared, connected coaxially, and the hollow formed by a succession of such annular structures can serve as the lumen. In other words, the annular smooth muscle tissue is arranged without gaps in a plane perpendicular to the central axis of the artificial tissue tube. By winding the smooth muscle tissue helically or annularly in this way, the smooth muscle cells constituting the smooth muscle can be aligned circumferentially around the artificial tissue tube. As a result, the artificial tissue tube can contract and relax more flexibly at the tissue and cellular levels, enabling more faithful replication of deformation reactions within the body, such as those of blood vessels.

The artificial tissue tube of the invention preferably further comprises an extracellular matrix (ECM) layer containing extracellular matrix material around the outer periphery of the smooth muscle layer. Examples of extracellular matrix materials include collagen, proteoglycans, fibronectin, and laminin. The extracellular matrix layer serves to fill the space around the smooth muscle layer and acts as a physical support and a scaffold for adhesion between cells and substrate.

The artificial tissue tube preferably also includes endothelial cells on the inner wall of the smooth muscle layer. Endothelial cells are monolayered cells covering the luminal surface of smooth muscle in blood vessels. Forming a layer of endothelial cells on the inner wall of the smooth muscle layer enables reproduction of biological structures such as blood vessels. The artificial tissue tube can be used not only as an artificial blood vessel but also as an artificial bronchus or artificial intestine, where contraction and relaxation are required.

A second aspect of the present invention relates to a method for manufacturing the artificial tissue tube. This method fundamentally pertains to the manufacture of the artificial tissue tube described in the first aspect. The manufacturing method includes a smooth muscle layer formation step in which fibrous smooth muscle tissue is wound around a mandrel to produce a smooth muscle layer. That is, by winding fibrous smooth muscle tissue helically or annularly around the mandrel, a smooth muscle layer is formed around the mandrel. Afterward, removing the mandrel yields a tubular artificial tissue tube containing the smooth muscle layer.

In the manufacturing method, the smooth muscle layer formation step is preferably carried out by tilting the mandrel and helically winding the fibrous smooth muscle tissue around it. In this way, the smooth muscle layer can be formed efficiently and simply.

The manufacturing method preferably further includes a step of forming the fibrous smooth muscle tissue. This step includes: a mandrel placement step in which the mandrel is placed in a first groove of a mold for smooth muscle tissue formation; a liquid injection step in which a liquid containing smooth muscle cells is injected into a second groove intersecting the first groove; and a smooth muscle tissue acquisition step in which the smooth muscle cells are cultured after the liquid injection step to obtain fibrous smooth muscle tissue. During this process, part of the smooth muscle tissue becomes bonded to the mandrel. Thus, fibrous smooth muscle tissue partially attached to the mandrel can be obtained. Subsequently, the fibrous smooth muscle tissue can be wound around the mandrel as described.

The manufacturing method preferably further includes an extracellular matrix layer formation step. After forming the smooth muscle layer, the smooth muscle layer is immersed in a liquid containing extracellular matrix material to form the extracellular matrix layer.

The manufacturing method preferably further includes an endothelial cell seeding step, in which endothelial cells are seeded onto the inner wall of the smooth muscle layer. This results in the formation of a layer of endothelial cells on the inner wall of the smooth muscle layer.

### Advantageous Effects of Invention

According to the present invention, it is possible to provide an artificial tissue tube capable of more flexible deformation. Thus, the physiological deformation reactions of biological structures such as blood vessels can be more faithfully reproduced.

### BRIEF DESCRIPTION OF THE DRAWINGS

FIG. 1 illustrates an artificial tissue tube according to an embodiment of the present invention.

FIG. 2 shows a cross-sectional structure of the artificial tissue tube according to the embodiment.

FIG. 3 schematically represents a process for forming a smooth muscle layer in the artificial tissue tube.

FIG. 4 shows examples of a first and second mold and a base used for manufacturing the artificial tissue tube.

FIG. 5 illustrates an example process of manufacturing fibrous smooth muscle tissue.

FIG. 6 shows an example manufacturing process for the artificial tissue tube.

FIG. 7 illustrates an example method for determining the inclination angle of the mandrel during manufacture of the artificial tissue tube.

FIG. 8 shows a modification example of the first mold used for manufacturing the artificial tissue tube.

FIG. 9 presents a substitute photograph and graph evaluating alignment of smooth muscle cells (SMCs) in the fiber. FIG. 9(a) is a projection image of the z-stack confocal fluorescence image of actin filaments in HUASMCs stained with a live cell actin probe. FIG. 9(b) is an enlarged image of FIG. 9(a). FIG. 9(c) is a graph showing the results of image analysis related to the orientation of actin filaments within the HUASMC fibers.

FIG. 10 presents a substitute photograph and graph showing manufacturing results of an SMC-embedded collagen tube made with HUASMC fibers. FIG. 10(a) is a photograph showing the overall image of a coiled SMC-embedded collagen tube. FIG. 10(b) is a photograph replacing the phase contrast image of the tube. FIG. 10(c) is a photograph replacing the projection image of the z-stack confocal fluorescence image of actin stained in the SMCs within the tube. FIG. 10(d) is a graph replacing the diagram showing the orientation of actin filaments in HUASMCs.

### DESCRIPTION OF EMBODIMENTS

Hereinafter, embodiments for carrying out the present invention will be described with reference to the drawings. The present invention is not limited to the embodiments described below and also encompasses appropriate modifications that would be apparent to those skilled in the art based on the following embodiments.

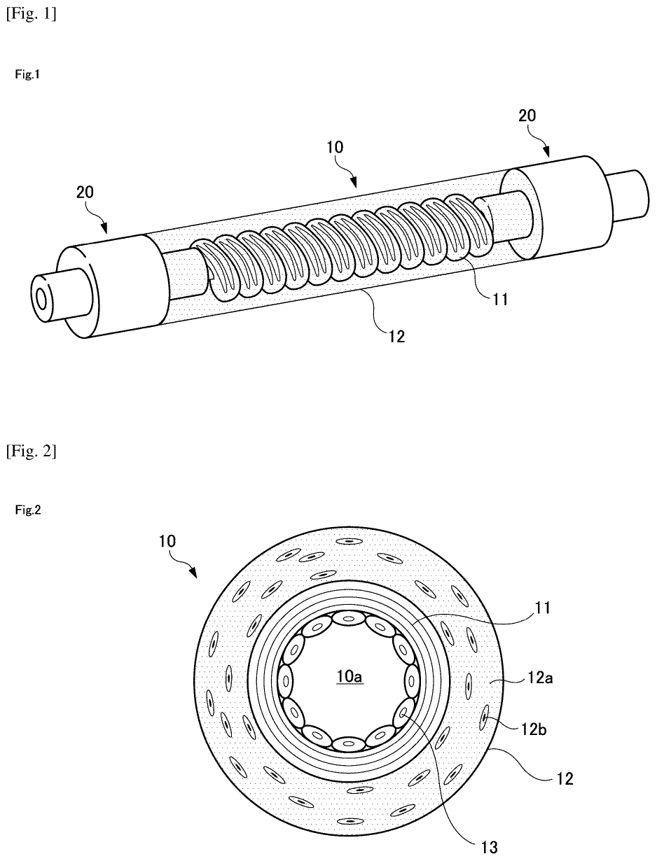

FIG. 1 shows an artificial tissue tube 10 according to an embodiment of the present invention, and FIG. 2 shows the cross-sectional structure of the artificial tissue tube 10. As shown in these figures, the artificial tissue tube 10 fundamentally has a hollow structure with an annular cross-section, and a hollow portion 10a is formed along its central axis. The artificial tissue tube 10 has a multilayer structure when viewed in cross-section. Specifically, the artificial tissue tube 10 of the present embodiment includes a smooth muscle layer 11, an extracellular matrix layer 12, and an endothelial cell layer 13, forming a three-layer structure. The extracellular matrix layer 12 is laminated on the outer peripheral surface of the smooth muscle layer 11, and the endothelial cell layer 13 is laminated on the inner peripheral surface of the smooth muscle layer 11. The cross-sectional shape of the artificial tissue tube 10 is not particularly limited and may be circular as shown in FIG. 2, or it may be polygonal, such as elliptical, triangular, or quadrangular.

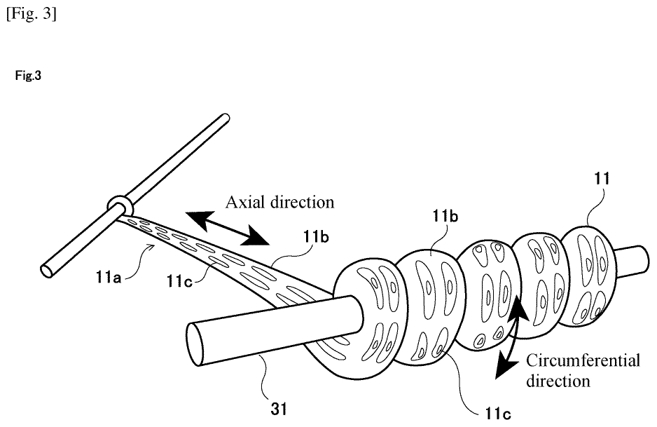

FIG. 3 schematically illustrates the method for forming the smooth muscle layer 11 in the artificial tissue tube 10 of the present embodiment. As shown in FIG. 3, the smooth muscle layer 11 can be formed by helically and tightly winding fibrous smooth muscle tissue 11a around a mandrel 31. The smooth muscle tissue 11a is composed of an extracellular matrix material 11b, such as collagen, and smooth muscle cells 11c. The smooth muscle tissue 11a may also include fibroblasts. In the fibrous state before being wound as shown in FIG. 3, the smooth muscle cells 11c are aligned along the axial (longitudinal) direction of the smooth muscle tissue 11a. By winding such axially aligned smooth muscle tissue 11a helically around the mandrel 31, the smooth muscle layer 11 of the artificial tissue tube 10 is formed. In the state where the smooth muscle tissue 11a is helically wound to form the smooth muscle layer 11, the smooth muscle cells 11c are aligned circumferentially around the artificial tissue tube 10. Typically, in blood vessels in the body, smooth muscle cells are aligned circumferentially around the vessel and repeatedly contract and relax. Therefore, by aligning the smooth muscle cells 11c circumferentially in the artificial tissue tube 10 according to the present invention, the tube can contract and relax flexibly in immune responses, similarly to human blood vessels. As a result, deformation reactions of blood vessels in the body can be more faithfully reproduced.

From this perspective, as long as the smooth muscle cells 11c in the smooth muscle layer 11 are aligned circumferentially relative to the artificial tissue tube 10, the deformation reactions inside the body can be replicated. Therefore, the smooth muscle layer 11 is not limited to one formed by helically winding smooth muscle tissue 11a. For example, the smooth muscle layer 11 may be formed by arranging multiple rings of smooth muscle tissue 11a of the same diameter along the axis of the artificial tissue tube 10. That is, in an annularly wound smooth muscle tissue 11a, the smooth muscle cells 11c inside are also aligned circumferentially relative to the tube 10. By connecting multiple such annular smooth muscle tissues 11a along the artificial tissue tube 10, it is possible to realize deformation reactions very similar to those of blood vessels in the body.

The extracellular matrix material 11b forming the smooth muscle layer 11 is not particularly limited. Examples include collagen, laminin, gelatin, cadherin, hyaluronic acid, fibronectin, fibrillin, elastin, chitin, chitosan, vitronectin, and proteoglycans. Among these, collagen is preferably used. The extracellular matrix material 11b may be used alone or in combination of two or more types.

As shown in FIG. 2, the extracellular matrix layer 12 is a layer laminated on the outer peripheral surface of the smooth muscle layer 11 and includes, for example, an extracellular matrix material 12a and adventitial cells 12b such as fibroblasts. The examples of the extracellular matrix material 12a forming the extracellular matrix layer 12 are the same as those of the extracellular matrix material 11b forming the smooth muscle layer 11 and will not be described again.

As shown in FIG. 2, the endothelial cells 13 are present on the inner peripheral surface of the smooth muscle layer 11, forming a layer through close junctions between multiple endothelial cells 13. The endothelial cell layer can be formed by seeding endothelial cells 13 onto the inner wall surface after forming the tubular smooth muscle layer 11.

As shown in FIG. 1, one or both ends of the artificial tissue tube 10 may be provided with a connector 20 for connecting the artificial tissue tube 10 to another tube or similar structure. The connector 20 is also tubular in structure, similar to the artificial tissue tube 10, and is connected to the end of the artificial tissue tube 10 such that their respective hollow portions are in communication. The connector 20 can be designed to match the shape and dimensions of the artificial tissue tube 10. The material constituting the connector 20 can be selected from known biocompatible materials, such as siliconeresin, fluororesin, polyethylene, polypropylene, or other materials like metals, glass, ceramics, etc.

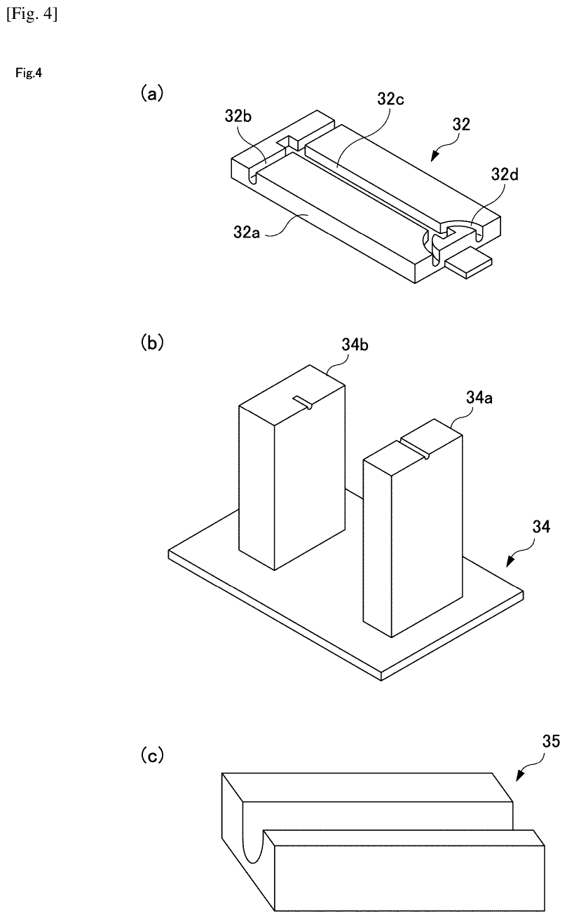

Next, with reference to FIGS. 4 to 7, the method for manufacturing the artificial tissue tube 10 will be explained in detail. FIG. 4 shows an example of the apparatus used to manufacture the artificial tissue tube 10. FIG. 4(a) shows the first mold 32 used to create the fibrous smooth muscle tissue 11a, FIG. 4(b) shows the base 34 used when the smooth muscle tissue 11a is wound around the mandrel 31, and FIG. 4(c) shows the second mold 35 used to form the extracellular matrix layer 12 on the outer circumferential surface of the smooth muscle layer 11.

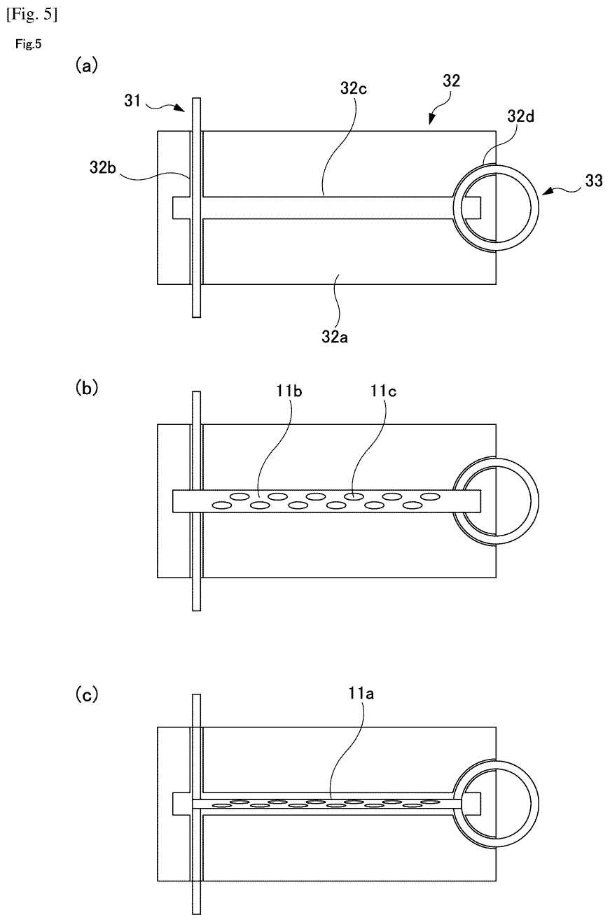

FIG. 5 shows the process of creating the fibrous smooth muscle tissue 11a using the first mold 32, as shown in FIG. 4(a). As illustrated in both FIG. 4(a) and FIG. 5(a), the first mold 32 is composed of a flat substrate 32a with a certain thickness, on which the first groove 32b, the second groove 32c, and the third groove 32d are formed. The first groove 32b intersects the second groove 32c at one end of the second groove 32c, and the third groove 32d intersects the second groove 32c at the other end of the second groove 32c.

The first groove 32b is intended for placing the mandrel 31, around which the fibrous smooth muscle tissue 11a is wound. The thickness (diameter) of the mandrel 31 roughly corresponds to the diameter of the hollow portion 10a of the artificial tissue tube 10, so an appropriately sized mandrel 31 should be selected depending on the intended application of the artificial tissue tube 10. For example, when manufacturing an artificial tissue tube 10 for use as an artificial blood vessel, the thickness of the mandrel 31 may be set to approximately 0.1 to 10 mm. If the goal is to obtain an artificial vessel for an aorta, the thickness of the mandrel 31 can be set to 10 to 30 mm. The first groove 32b only needs to accommodate a mandrel 31 of such thickness, and a clearance of about 0.01 to 0.1 mm may be provided between the groove and the mandrel. The second groove 32c is for injecting a solution used to form the smooth muscle tissue 11a. The third groove 32d is for placing a weight 33 that attaches to the smooth muscle tissue 11a. The weight 33 is used to apply a vertical load to the smooth muscle tissue 11a during winding around the mandrel 31. The shape of the weight 33 is not particularly limited; as shown in FIG. 5, it may be annular, polygonal, or rod-shaped-either straight or curved. The weight of the weight 33 is also not specifically restricted, as long as it provides an appropriate load to the smooth muscle tissue 11a. A clearance of about 0.01 to 0.1 mm may be provided between the third groove 32d and the weight 33. In this way, the various grooves 32b, 32c, and 32d of the first mold 32 are designed such that one end of the fibrous smooth muscle tissue 11a is connected to the mandrel 31, while the other end is attached to the weight 33.

For production, first, as shown in FIG. 5(a), the mandrel 31 is placed in the first groove 32b, and the weight 33 is placed in the third groove 32d.

The materials for the mandrel 31 and the weight 33 are not particularly limited but are preferably metals commonly used in medical devices, such as tungsten, stainless steel, titanium, aluminum, or niobium.

Next, as shown in FIG. 5(b), a solution for forming the smooth muscle tissue 11a is injected into the second groove 32c and solidified. This solution is typically a sol or gel containing an extracellular matrix material 11b (e.g., collagen) and smooth muscle cells 11c. Specifically, a hydrogel of collagen in which smooth muscle cells 11c are suspended can be used. Upon injection, the solution comes into contact with the mandrel 31 and the weight 33 installed at both ends of the second groove 32c. To prevent the solution from leaking into the first groove 32b and the third groove 32d, the clearance between the grooves should be appropriately adjusted. The injected solution is then incubated, for example, at 35-40° C., to solidify.

Next, as shown in FIG. 5(c), while maintaining the state in which the solidified solution in the second groove 32c is in contact with both the mandrel 31 and the weight 33, the smooth muscle cells 11c are immersed in a culture medium and cultured. At this time, the entire first mold 32 may also be immersed in the culture medium. The culture conditions for the smooth muscle cells 11c are not particularly limited; however, in general, culturing is completed by leaving the cells to stand at a temperature of from 35° C. to 40° C. for 12 to 48 hours. Once culturing is complete, a dense fibrous smooth muscle tissue 11a containing both extracellular matrix material 11b and smooth muscle cells 11c is obtained. As a result, one end of the smooth muscle tissue 11a is bound to the mandrel 31, while the other end is bound to the weight 33.

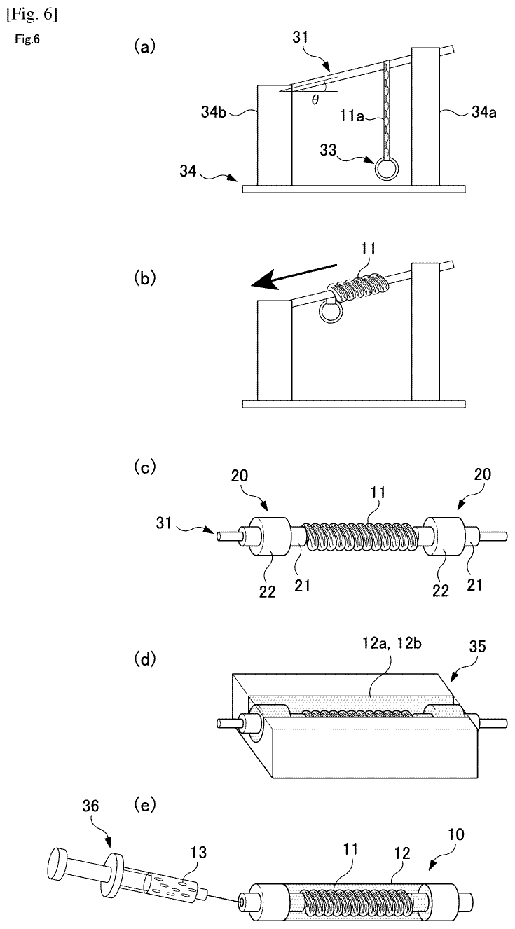

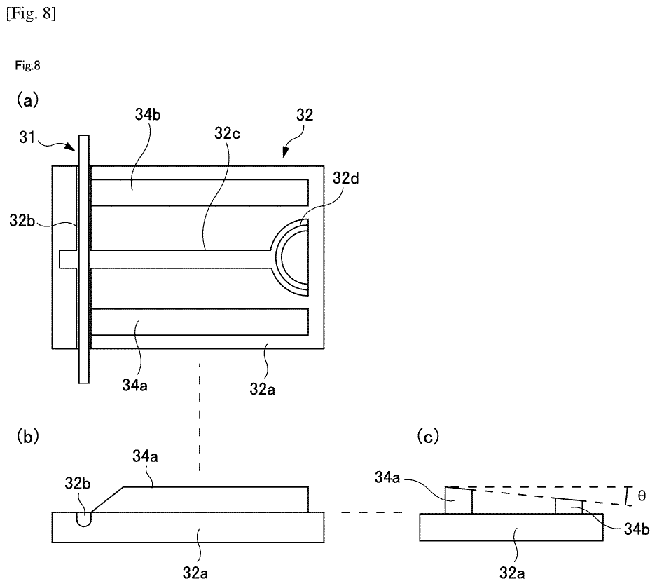

FIG. 6 shows the process of manufacturing the artificial tissue tube 10 using the fibrous smooth muscle tissue 11a obtained as described above. First, as shown in FIG. 6(a), the mandrel 31 with the attached smooth muscle tissue 11a is placed onto a base 34. As shown in FIGS. 4(a) and 6(b), the base 34 has a first support 34a and a second support 34b. The first support 34a is designed to hold the mandrel 31 at a height higher than the second support 34b. Thus, when the mandrel 31 is placed bridging the first and second supports 34a and 34b, the mandrel 31 is tilted downward from the first support 34a toward the second support 34b. In addition, the smooth muscle tissue 11a has the weight 33 attached to it. Therefore, when the mandrel 31 is placed on the first and second supports 34a and 34b, the smooth muscle tissue 11a hangs straight down under the vertical load of the weight 33. When setting up the mandrel 31, the heights of the first and second supports 34a and 34b should be adjusted so that the weight 33 attached to the smooth muscle tissue 11a does not touch the ground or the base 34. Moreover, this step can also be performed by equipping the first mold 32 (FIG. 4(a)) used for creating the fibrous smooth muscle tissue 11a with an integrated support structure (the first and second supports 34a and 34b), and rolling the mandrel 31 over this support structure. An example of such a modified mold shape is shown in FIG. 8.

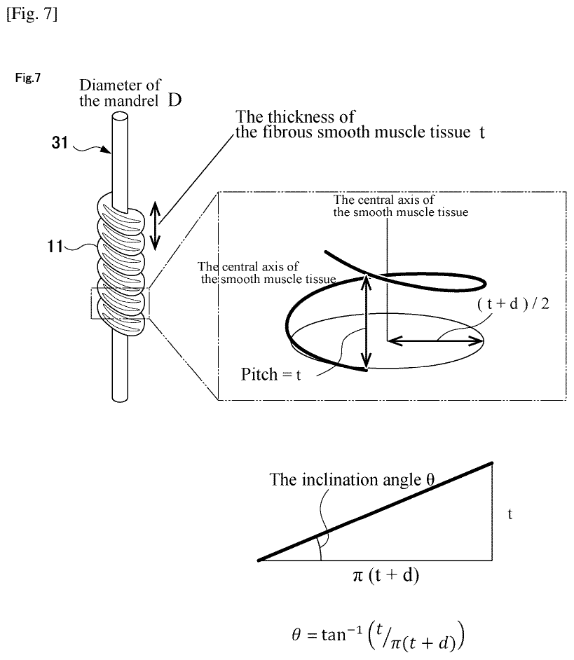

In FIGS. 6 and 8, the inclination angle of the mandrel 31 placed on the first and second supports 34a and 34b is indicated by the symbol θ. The inclination angle θ of the mandrel 31 can be adjusted by changing the height of the first and second supports 34a and 34b. A method for determining the appropriate inclination angle θ is illustrated in FIG. 7. As shown in FIG. 7, the appropriate inclination angle θ of the mandrel 31 depends on the thickness of the smooth muscle tissue 11a and the diameter of the mandrel 31. Specifically, if the thickness of the smooth muscle tissue 11a is t and the diameter of the mandrel 31 is d, the appropriate inclination angle θ of the mandrel 31 can be calculated by the following formula:

$$θ = {tan}^{- 1} ( t / π ( t + d ) ) \qquad [ Formula 1 ]$$

Next, as shown in FIG. 6(b), the mandrel 31 placed on the first and second supports 34a and 34b is rotated around its axis to wind the fibrous smooth muscle tissue 11a onto the mandrel 31. If the inclination angle θ of the mandrel 31 is appropriately set according to the above formula, the smooth muscle tissue 11a can be helically wound around the mandrel 31 without overlapping or creating gaps. Since a vertical load is applied to the smooth muscle tissue 11a by the weight 33, the tissue is stretched in the axial direction while being wound onto the mandrel 31. In this way, the smooth muscle layer 11 is formed around the mandrel 31.

Next, as shown in FIG. 6(c), with the smooth muscle tissue 11a wound around the mandrel 31, the weight 33 is removed, and connectors 20 are attached at both ends of the mandrel 31 to sandwich the smooth muscle layer 11 between them. Each connector 20 includes a small-diameter tubular glass capillary 21 and a larger-diameter silicone tube 22. The glass capillary 21 can be inserted into the hollow portion of the silicone tube 22, and the mandrel 31 can be inserted into the hollow portion of the glass capillary 21. Since the hollow portion of the glass capillary 21 and the smooth muscle layer 11 both have the mandrel 31 inserted through them, they are interconnected.

Next, as shown in FIG. 6(d), the smooth muscle layer 11 and the connector 20, while still mounted on the mandrel 31, are placed in the groove of the second mold 35. A gel-like or sol-like solution containing an extracellular matrix material 12a such as collagen and Adventitial cells 12b such as fibroblasts is then injected into the groove of the second mold 35. At this stage, at minimum, the smooth muscle layer 11 formed on the mandrel 31 is immersed in the solution. Additionally, the connector 20 may also be immersed in the solution together with the smooth muscle layer 11. In this state, the Adventitial cells 12b contained in the solution are cultured. There are no particular restrictions on the culture conditions for the Adventitial cells 12b; generally, culturing is completed by standing the solution at a temperature of 35 to 40° C. for 12 to 48 hours. Once culturing is complete, the initially gel-like or sol-like solution solidifies, and an extracellular matrix layer 12 containing the extracellular matrix material 12a and the Adventitial cells 12b is formed around the smooth muscle layer 11. This extracellular matrix layer 12 integrates the smooth muscle layer 11 and the connector 20 into a unified structure.

Next, as shown in FIG. 6(d), after the extracellular matrix layer 12 has solidified, the mandrel 31 is withdrawn. As a result, the region previously occupied by the mandrel 31 becomes a cavity. This cavity is in communication with both the connector 20 and the smooth muscle layer 11, and functions, for example, as the lumen of an artificial blood vessel. Endothelial cells 13 are then seeded into the hollow portion formed by removing the mandrel 31, using, for instance, a syringe 36. As a result, a layer of endothelial cells 13 is formed primarily on the inner circumferential surface of the smooth muscle layer 11. In this manner, an artificial tissue tube 10 is obtained in which the endothelial cell layer 13, the smooth muscle layer 11, and the extracellular matrix layer 12 are laminated in that order. As explained with reference to FIG. 3, in the artificial tissue tube 10 thus fabricated, the smooth muscle cells 11c constituting the smooth muscle layer 11 are oriented circumferentially with respect to the tube. Therefore, the artificial tissue tube 10 can contract and relax flexibly, much like a natural blood vessel in the human body, and can more faithfully reproduce vascular deformation responses in vivo.

### Example 1

The invention will now be described in more detail through examples.

### Design of Molds and Base

For the fabrication of the artificial tissue tube, a first mold for forming smooth muscle tissue (FIG. 4(a)), a base for winding the smooth muscle tissue (FIG. 4(b)), and a second mold for forming the extracellular matrix layer (FIG. 4(c)) were prepared. Each mold and the base were made of polydimethylsiloxane (PDMS) or polylactic acid (PLA). The inclination angle of the mandrel was determined based on the thickness of the smooth muscle fiber according to Formula 1 described above.

### Preparation of Cell Suspension Pre-Gel Solution

Human umbilical artery smooth muscle cells (HUASMCs) were used as the smooth muscle cell model in this example. Human umbilical vein endothelial cells (HUVECs) were used as the endothelial cell model. HUASMCs and HUVECs are among the most common cell models used for in vitro experiments because umbilical veins are relatively accessible compared to other human blood vessels. Commercially available HUASMCs, which were isolated from donor umbilical veins and cryopreserved, were used. The culture medium used was Smooth Muscle Cell Growth Medium (SMCGM) supplemented with a bullet kit for HUASMCs and Endothelial Growth Medium-2 (EGM-2) for HUVECs. For co-culture of HUASMCs and HUVECs, a 1:1 volume ratio mixture of SMCGM and EGM-2 was used. Prior to seeding, the cells were cultured in dishes at 37° C. and 5% CO₂ under humidified conditions, with the medium changed every three days. To suspend HUASMCs in a collagen pre-gel solution, the cells were first detached from the dish using trypsin-EDTA treatment. Subsequently, the trypsin-treated HUASMCs were suspended in the collagen pre-gel solution immediately before the fabrication process. To seed HUVECs on the collagen surface, trypsin-treated HUVECs were suspended in the medium and the resulting cell suspension was directly injected into the hollow portion of the device.

### [Formation of Smooth Muscle Fibers]

The method for producing a HUASMC tube can be divided into two main steps: a step of forming smooth muscle fibers, and a step involving the winding and coating to obtain a collagen tube embedded with a smooth muscle layer. The details of the smooth muscle fiber formation step are as follows:
[1] A straight tungsten wire (used as the core rod) and a circular tungsten wire (used as the weight) were placed and embedded in a first molding die for fiber formation (see FIG. 5(a)).
[2] A collagen pre-gel solution (1.0 or 2.0 mg/mL) in which HUASMCs (0.8 or 1.0×10⁶ cells/mL) were suspended was poured into the first mold and incubated at 37° C. to allow it to solidify (see FIG. 5(b)).
[3] The resulting tissue was cultured in SMCGM medium for 24 hours to induce cell-driven contraction and axial alignment, thereby forming a dense fiber (see FIG. 5(c)).

### [Fabrication of Smooth Muscle Layer-Embedded Vascular Model]

The dense smooth muscle fibers obtained as described above were wound around a core rod and embedded in collagen gel to fabricate a flexible, deformable collagen tube device in which smooth muscle cells are circumferentially aligned. Prior to winding, the inclination angle of the winding stage was designed based on the obtained smooth muscle fibers. To wind the fibers without any gaps, the helical pitch of the wound fiber must match the fiber thickness. The inclination angle θ of the core rod was calculated using the previously described Equation 1. The detailed steps for fabricating the collagen tube device are as follows:
[4] The molded smooth muscle fibers were removed and fixed to the covered winding base (FIG. 6(a)).
[5] The tungsten wire (mandrel) was rotated to wind the smooth muscle fibers around it (FIG. 6(b)).
[6] Connectors composed of a silicone tube and a glass capillary were inserted from both sides onto the tungsten wire (FIG. 6(c)).
[7] Using the second mold, a collagen pre-gel solution (4 mg/mL) was poured around the smooth muscle layer and connectors, and the device was coated with collagen. The setup was then incubated at 37° C. to solidify the collagen (FIG. 6(d)).
[8] The second mold was removed, yielding a collagen tube with circumferentially aligned smooth muscle layers (FIG. 6(e)). By seeding endothelial cells inside the tube, an arterial tissue model was obtained.

### [Fluorescent Staining and Observation]

To evaluate the alignment of actin fibers, live-cell actin probes (SPY-555 actin, Spirochrome) were used for staining. The fabricated tissue was immersed in a diluted SPY-555 actin solution (1/1,000,000) and incubated for 1 hour to stain the actin of HUA-SMCs. For time-lapse imaging, time-lapse sequence images were generated by aligning the upper edge of the trimmed images of the HUA-SMC layer, allowing for easy comparison of contraction behavior.

### [Evaluation of Axial Alignment of Smooth Muscle Fibers]

To evaluate the alignment of smooth muscle fibers, actin filaments in the smooth muscle were stained using a live-cell actin probe. Fluorescence microscopy images at high magnification clearly showed the axial alignment of smooth muscle actin. The orientation of the actin filaments was analyzed by extracting actin lines from the fluorescence images using image analysis software. A histogram of the proportion of actin filaments in each direction demonstrated the alignment of HUA-SMCs along the fiber axis, where 0° was defined as the fiber direction. The mean orientation and variance were 0.21° and 7.88°, respectively, confirming that the fiber formation process reliably induced axial alignment of the smooth muscle.

### [Results of Fabrication of Smooth Muscle Layer-Embedded Collagen Tube]

Next, the fibrous smooth muscle tissue was rolled and embedded in collagen gel to create a collagen tube device with a smooth muscle layer arranged circumferentially inside. Fibers approximately 300 μm in diameter were used in the construction of the tube device. Using the formula mentioned above, the winding angle of the roll was determined to be 7°. The collagen tube, with smooth muscle fibers wound inside, was easily and stably fabricated. Phase contrast imaging confirmed the presence of smooth muscle fibers wound within the collagen tube. Live imaging of the actin filaments of the wound smooth muscle cells revealed that they were aligned circumferentially, which was visually confirmed. Additionally, actin lines were extracted from fluorescence images using image analysis software to analyze the directionality of the actin filaments. The histogram of the proportions of actin filaments in each direction confirmed that the fibers were aligned perpendicular to the tube direction. The tube direction was defined as 0°, and the average direction was found to be 80.8°, with a variance of 9.82°. The average direction of SMCs in five processed tubes was calculated to be 78.95±1.79°. Since the winding base was inclined at 7° with tungsten wire, the direction of the actin in the wound smooth muscle was approximately 83°. This demonstrates that the proposed manufacturing method effectively achieves circumferential alignment of the smooth muscle cells in the tube device. Furthermore, the average direction had a coefficient of variation of 2.2%, confirming the stable production of a smooth muscle layer aligned circumferentially. From the perspective of mimicking in vivo tissue, it is known that smooth muscle in native tissues forms a helical structure with a wide range of helical pitch angles (from below 30° to above) 70°. These angles vary depending on factors such as vessel type, diameter, and blood pressure. Compared to in vivo values, the smooth muscle in this proposed model exhibits a smaller helical pitch angle. However, since the thickness of the smooth muscle fibers and the winding angle can be controlled, it is believed that adjusting the manufacturing conditions could allow for control over the pitch angle.

### Example 2

### Evaluation of Axial Alignment of Smooth Muscle Cell (SMC) Fibers

To evaluate the alignment of SMC fibers, the actin filaments within the SMCs were stained using a live-cell actin probe. The axial alignment of the actin in the SMCs was visually observed from the magnified fluorescence microscope images. The directionality of the actin filaments was analyzed by extracting the actin lines from the fluorescence images using image analysis software.

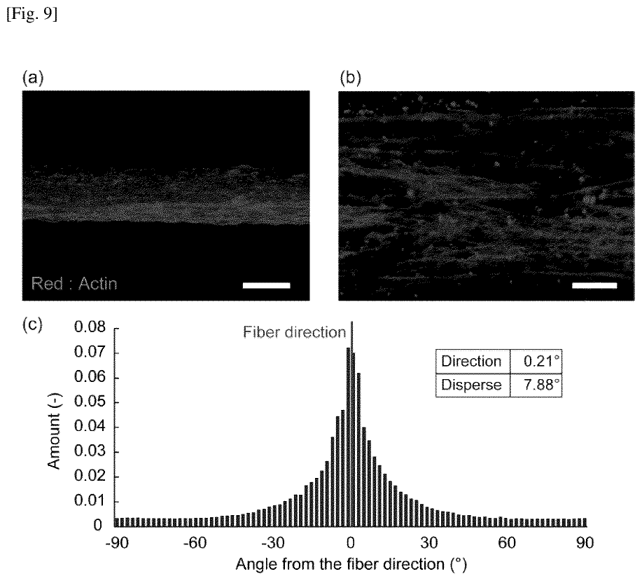

FIG. 9 shows photographs and graphs that evaluate the alignment of smooth muscle cells (SMC) fibers. FIG. 9(a) is a projection image of z-stack confocal fluorescence images of the actin filaments in HUASMCs stained with a live-cell actin probe. The scale bar is 200 μm. FIG. 9(b) is an enlarged version of FIG. 9(a). The scale bar is 10 μm. From FIGS. 9(a) and (b), axial alignment is observed. FIG. 9(c) shows a graph of the image analysis results regarding the directionality of actin filaments in HUASMC fibers. The horizontal axis indicates the angle relative to the fiber alignment direction, with 0° representing the fiber direction. The vertical axis indicates the distribution amount (ratio). The histogram of the ratio of actin filaments in each direction shows the alignment of HUASMCs in the fiber direction. The average direction and variance were 0.21° and 7.88°, respectively. These values confirm the axial alignment of SMCs due to the fiber formation process.

### Example 3

### Fabrication Results of SMC Layer-Embedded Collagen Tubes

Next, the formed fibrous SMC tissue was wound and embedded in collagen gel to produce a collagen tube device with an SMC layer aligned circumferentially inside. Fibers with a thickness of approximately 300 μm were used for the fabrication of the tube device. Therefore, by substituting values into equation (Formula 1), the inclination angle of the winding mold was determined to be 7°.

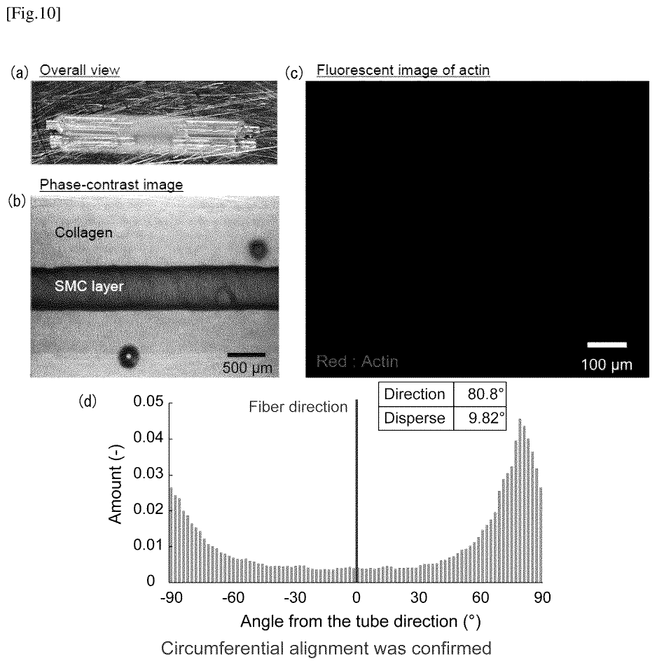

FIG. 10 shows the results of manufacturing an SMC layer-embedded collagen tube made from HUASMC fibers, represented by photographs and graphs instead of diagrams. FIG. 10(a) is a photograph showing the overall view of the wound SMC-embedded collagen tube. FIG. 10(b) is a photograph representing the phase contrast image of the tube. The scale bar is 500 μm. FIG. 10(c) is a photograph showing the projection image of the z-stack confocal fluorescence image of the stained actin in the SMCs within the tube. The scale bar is 100 μm. From FIG. 10(c), circumferential alignment is observed.

As shown in FIG. 10(a), the collagen tube was easily and stably manufactured. The phase contrast image in FIG. 10(b) clearly revealed the presence of wound SMC fibers within the collagen tube. From FIG. 10(c), live imaging of actin demonstrated the observation of the actin filaments in the wound SMCs, and it was visually confirmed that they were aligned circumferentially.

Again, the orientation of the actin filaments was analyzed by extracting actin lines from the fluorescence images using image analysis software.

FIG. 10(d) is a graph representing the orientation of actin filaments in HUASMCs, serving as an alternative to a drawing. The horizontal axis indicates the angle from the fiber direction, where 0° corresponds to the tube direction. From FIG. 10(d), it is evident that the average orientation of the filaments is perpendicular to the tube direction, indicating circumferential alignment of the SMC tissue.

The histogram of the actin filament orientation ratios showed alignment in the direction perpendicular to the tube axis. In the sample, the mean and variance of the filament orientation were 80.8° and 9.82°, respectively, and the average of the mean orientations of SMCs in five fabricated tubes was calculated to be 78.95±1.79°. Since the winding mold had a 7° tilt applied to the tungsten wire, the orientation of the wound SMC actin filaments was expected to be approximately 83°. Therefore, the results shown in FIG. 10(d) indicate that the fabrication method successfully achieved circumferential alignment of SMCs within the tube device. Furthermore, the average orientation exhibited a coefficient of variation of 2.2%, confirming that the circumferentially aligned SMC layer was produced with high reproducibility.

From the perspective of biomimicry, SMCs in native tissues are known to form helical structures with a wide range of helical pitch angles (from below 30° to above) 70°. These angles vary depending on factors such as the type, thickness, and blood pressure of the vessel, among others. Compared with the values observed in vivo, the proposed model exhibits a smaller helical pitch angle. However, the helical pitch angle may be controllable by adjusting the fabrication conditions, since the above results demonstrate that both the thickness of the SMC fibers and the winding angle can be regulated.

In the present specification, embodiments and examples of the invention have been described with reference to the drawings in order to illustrate the technical features of the invention. However, the invention is not limited to the embodiments and examples described above, and includes obvious modifications and improvements that can be made by those skilled in the art based on the contents disclosed herein.

### REFERENCE SIGNS LIST

10: Artificial tissue tube
10a: Hollow portion
11: Smooth muscle layer
11a: Smooth muscle tissue
11b: Extracellular matrix material
11c: Smooth muscle cell
12: Extracellular matrix layer
12a: Extracellular matrix material
12b: Adventitial cell
13: Endothelial cell
20: Connector
21: Glass capillary
22: Silicone tube
31: Mandrel
32: First mold
32a: Substrate
32b: First groove
32c: Second groove
32d: Third groove
33: Weight
34: Base
34a: First support
34b: Second support
35: Second mold
36: Syringe

1. An artificial tissue tube comprising
a smooth muscle layer that includes a smooth muscle coiled in a spiral shape or an annular shape, wherein
muscle is a fibrous smooth n muscle tissue.

2. The artificial tissue tube according to claim 1, further comprising
an extracellular matrix layer that includes an extracellular matrix material present on an outer periphery of the smooth muscle layer.

3. The artificial tissue tube according to claim 2, comprising
endothelial cells on an inner wall of the smooth muscle layer.

4. The artificial tissue tube according to claim 1, wherein
the artificial tissue tube is capable of contracting and relaxing.

5. The artificial tissue tube according to claim 1, wherein
the artificial tissue tube is an artificial blood vessel.

6. A method for producing an artificial tissue tube, the method comprising
a smooth muscle layer preparation step of winding a fibrous smooth muscle tissue around a core rod to produce a smooth muscle layer.

7. The method for producing an artificial tissue tube according to claim 6, wherein
the smooth muscle layer preparation step is a step of winding the fibrous smooth muscle tissue in a spiral shape around the core rod in a state in which the core rod is tilted.

8. The method for producing an artificial tissue tube according to claim 6, further comprising
a smooth muscle tissue preparation step of preparing the fibrous smooth muscle tissue, wherein
the smooth muscle tissue preparation step includes:
a core rod installation step of installing the core rod in a first groove of a mold for smooth muscle tissue preparation;
a liquid injection step of pouring a liquid containing smooth muscle cells into a second groove that intersects
with the first groove of the mold for smooth muscle tissue preparation; and
a smooth muscle tissue acquisition step of, after the liquid injection step, culturing the smooth muscle cells to obtain the fibrous smooth muscle tissue.

9. The method for producing an artificial tissue tube according to claim 6, further comprising,
after the smooth muscle layer preparation step, an extracellular matrix layer formation step of immersing the smooth muscle layer in a liquid containing an extracellular matrix material to form an extracellular matrix layer.

10. The method for producing an artificial tissue tube according to claim 9, further comprising
an endothelial cell seeding step of seeding endothelial cells on an inner wall of the smooth muscle layer.

11. The artificial tissue tube according to claim 1, wherein
the smooth muscle tissue is oriented in a circumferential direction of the artificial tissue tube.

12. The artificial tissue tube according to claim 1, wherein
the smooth muscle is a smooth muscle that is coiled in a spiral shape.

13. An artificial tissue tube comprising
a smooth muscle layer that includes a smooth muscle that is coiled in a spiral shape.

14. An artificial tissue tube comprising
a smooth muscle layer that includes a smooth muscle coiled in a spiral shape or an annular shape, wherein
the smooth muscle includes a smooth muscle tissue oriented in a circumferential direction of the artificial tissue tube.
